(19) United States
(12) Patent Application Publication
(10) Pub. No.: US 2025/0169844 A1
(43) Pub. Date: May 29, 2025

(54) ULTRASONIC SURGICAL BLADE FOR USE WITH ULTRASONIC SURGICAL INSTRUMENTS

(71) Applicant: Cilag GmbH International, Zug (CH)

(72) Inventors: Jacob S. Gee, Cincinnati, OH (US); Amy L. Marcotte, Mason, OH (US); John A. Weed, III, Monroe, OH (US)

(21) Appl. No.: 18/898,058

(22) Filed: Sep. 26, 2024

Related U.S. Application Data
(63) Continuation of application No. 17/244,216, filed on Apr. 29, 2021, now Pat. No. 12,156,674.
Application No. 17/244,216 is a continuation of application No. 14/742,504, filed on Jun. 17, 2015, now Pat. No. 11,020,140.

(51) Int. Cl. A61B 17/32; A61B 17/00

(52) U.S. Cl. CPC A61B 17/320092; A61B 2017/00738; A61B 2017/320075; A61B 2017/320077; A61B 2017/320078; A61B 2017/320094; A61B 2017/320095

(57) ABSTRACT

A surgical instrument includes an ultrasonic transducer, an ultrasonic transmission waveguide extending from the ultrasonic transducer, and an ultrasonic blade acoustically coupled to the ultrasonic transmission waveguide. The ultrasonic blade includes a base and a curved body extending distally from the base. The curved body includes a tissue-treating surface extending on a first side of the curved body and a curved edge extending on a second side of the curved body opposite the first side. The curved edge has a proximal end and a distal end, wherein the proximal end is offset from the distal end in a first direction, wherein the proximal end is offset from the distal end in a second direction, and wherein the first direction is perpendicular to the second direction.

### BACKGROUND

This application is a continuation application claiming priority to 35 U.S.C. § 120 to U.S. patent application Ser. No. 17/244,216, entitled ULTRASONIC SURGICAL BLADE FOR USE WITH ULTRASONIC SURGICAL INSTRUMENTS, filed Apr. 29, 2021, now U.S. Patent Application Publication No. 2021/0315605, which claims priority to 35 U.S.C. § 120 to U.S. patent application Ser. No. 14/742,504, entitled ULTRASONIC SURGICAL BLADE FOR USE WITH ULTRASONIC SURGICAL INSTRUMENTS, filed Jun. 17, 2015, which issued Jun. 1, 2021, now U.S. Pat. No. 11,020,140, the entire disclosure of which is hereby incorporated by reference herein.

The present disclosure relates to surgical instruments and, in various embodiments, to ultrasonic surgical instruments.

Ultrasonic surgical instruments are used in many applications in surgical procedures by virtue of their unique performance characteristics. In various instances, ultrasonic surgical instruments can be configured for use in open, laparoscopic, or endoscopic surgical procedures. Ultrasonic surgical instruments can be also configured for use in robotic-assisted surgical procedures.

### FIGURES

The features of the various embodiments are set forth with particularity in the appended claims. The various embodiments, however, both as to organization and methods of operation, together with advantages thereof, may best be understood by reference to the following description, taken in conjunction with the accompanying drawings as follows:

FIG. 1 is a perspective view of an ultrasonic surgical blade according to one embodiment;

FIG. 2 is a perspective view of an ultrasonic surgical blade according to one embodiment;

FIG. 3 is a perspective view of an ultrasonic surgical blade according to one embodiment;

FIG. 4 is a perspective view of an ultrasonic surgical blade according to one embodiment;

FIG. 5 is a perspective view of an ultrasonic surgical blade according to one embodiment;

FIG. 6 is a perspective view of an ultrasonic surgical blade according to one embodiment;

FIG. 7 is a perspective view of an ultrasonic surgical blade according to one embodiment;

FIG. 8 is a perspective view of an ultrasonic surgical blade according to one embodiment;

FIG. 9 is a perspective view of an ultrasonic surgical blade according to one embodiment;

FIG. 9A is a front view of an ultrasonic surgical blade according to one embodiment;

FIG. 10 is a sectional view of the ultrasonic surgical blade shown in FIG. 12 taken along section line 10-10, according to one embodiment;

FIG. 11 is a top view of an ultrasonic surgical blade according to one embodiment;

FIG. 12 is a side view of an ultrasonic surgical blade according to one embodiment;

FIG. 13 is a sectional view of the ultrasonic surgical blade shown in FIG. 11 taken along section line 13-13, according to one embodiment;

FIG. 14 is a sectional view of the ultrasonic surgical blade shown in FIG. 11 taken along section line 14-14, according to one embodiment;

FIG. 15 is a sectional view of the ultrasonic surgical blade shown in FIG. 11 taken along section line 15-15, according to one embodiment;

FIG. 16 is a sectional view of the ultrasonic surgical blade shown in FIG. 11 taken along section line 16-16, according to one embodiment;

FIG. 17 is a sectional view of the ultrasonic surgical blade shown in FIG. 11 taken along section line 17-17, according to one embodiment;

FIG. 18 is a sectional view of the ultrasonic surgical blade shown in FIG. 11 taken along section line 18-18, according to one embodiment;

FIG. 19 is a sectional view of the ultrasonic surgical blade shown in FIG. 11 taken along section line 19-19, according to one embodiment;

FIG. 20 is a sectional view of the ultrasonic surgical blade shown in FIG. 11 taken along section line 20-20, according to one embodiment;

FIG. 21 is a sectional view of the ultrasonic surgical blade shown in FIG. 11 taken along section line 21-21, according to one embodiment;

FIG. 22 is a sectional view of the ultrasonic surgical blade shown in FIG. 11 taken along section line 22-22, according to one embodiment;

FIG. 23 is a partial side view of an ultrasonic surgical instrument including an end effector assembly according to one embodiment;

FIG. 24 is an exploded view of end effector assembly of the ultrasonic surgical instrument of FIG. 23 according to one embodiment;

FIG. 25 is a top view of an ultrasonic surgical blade according to one embodiment;

FIG. 26 is a sectional view of the ultrasonic surgical blade shown in FIG. 25 taken along section line 26-26, according to one embodiment;

FIG. 27 is a sectional view of the ultrasonic surgical blade shown in FIG. 25 taken along section line 27-27, according to one embodiment;

FIG. 28 is a sectional view of the ultrasonic surgical blade shown in FIG. 25 taken along section line 28-28, according to one embodiment;

FIG. 29 is a sectional view of the ultrasonic surgical blade shown in FIG. 25 taken along section line 29-29, according to one embodiment;

FIG. 30 is a sectional view of the ultrasonic surgical blade shown in FIG. 25 taken along section line 30-30, according to one embodiment;

FIG. 31 is a sectional view of the ultrasonic surgical blade shown in FIG. 25 taken along section line 31-31, according to one embodiment; and

FIG. 32 is a side view of an ultrasonic surgical instrument including an end effector assembly and a handle assembly according to one embodiment.

### DESCRIPTION

Numerous specific details are set forth to provide a thorough understanding of the overall structure, function, manufacture, and use of the embodiments as described in the specification and illustrated in the accompanying drawings. Well-known operations, components, and elements have not been described in detail so as not to obscure the embodiments described in the specification. The reader will understand that the embodiments described and illustrated herein are non-limiting examples, and thus it can be appreciated that the specific structural and functional details disclosed herein may be representative and illustrative. Variations and changes thereto may be made without departing from the scope of the claims.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a surgical system, device, or apparatus that “comprises,” “has,” “includes” or “contains” one or more elements possesses those one or more elements, but is not limited to possessing only those one or more elements. Likewise, an element of a system, device, or apparatus that “comprises,” “has,” “includes” or “contains” one or more features possesses those one or more features, but is not limited to possessing only those one or more features.

The terms “proximal” and “distal” are used herein with reference to a clinician manipulating the handle portion of the surgical instrument. The term “proximal” referring to the portion closest to the clinician and the term “distal” referring to the portion located away from the clinician. It will be further appreciated that, for convenience and clarity, spatial terms such as “vertical”, “horizontal”, “up”, and “down” may be used herein with respect to the drawings. However, surgical instruments are used in many orientations and positions, and these terms are not intended to be limiting and/or absolute.

Various exemplary devices and methods are provided for performing laparoscopic and minimally invasive surgical procedures. However, the reader will readily appreciate that the various methods and devices disclosed herein can be used in numerous surgical procedures and applications including, for example, in connection with open surgical procedures. As the present Detailed Description proceeds, the reader will further appreciate that the various instruments disclosed herein can be inserted into a body in any way, such as through a natural orifice, through an incision or puncture hole formed in tissue, etc. The working portions or end effector portions of the instruments can be inserted directly into a patient's body or can be inserted through an access device that has a working channel through which the end effector and elongated shaft of a surgical instrument can be advanced.

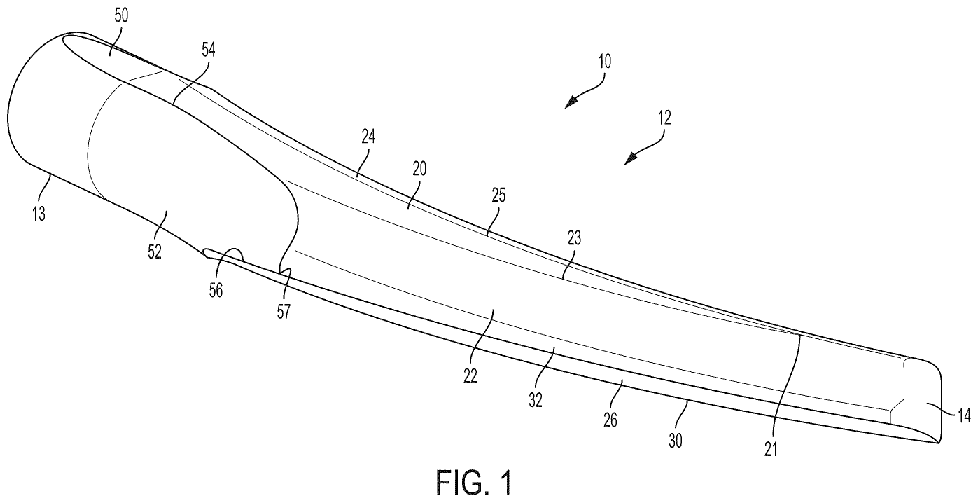

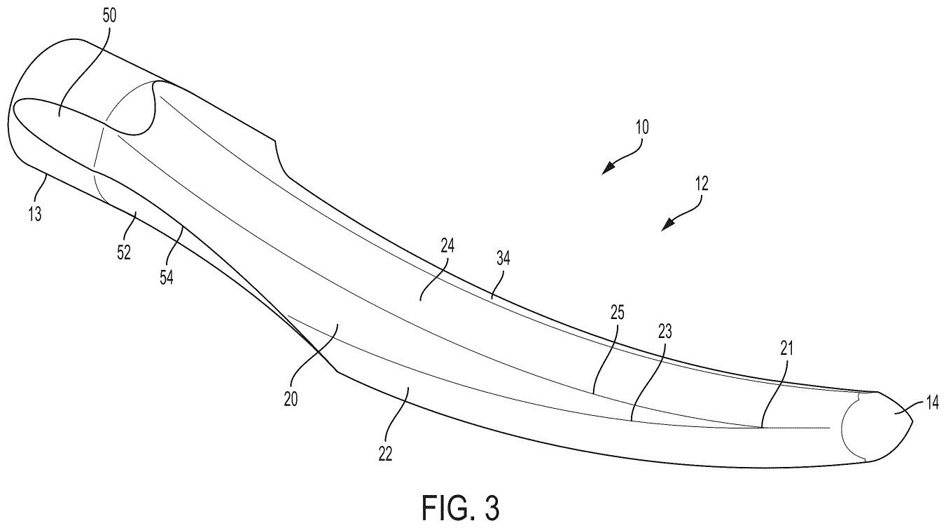

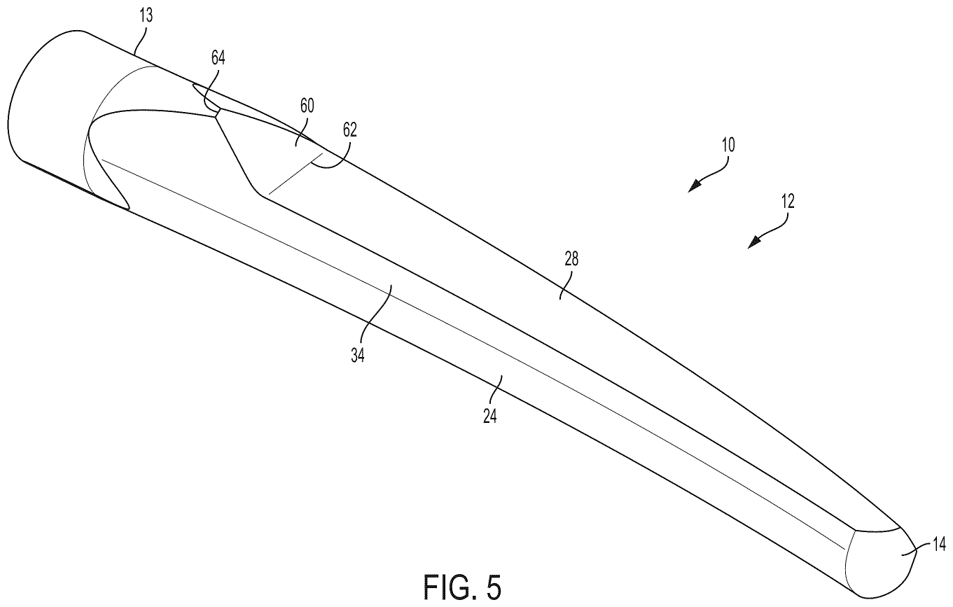

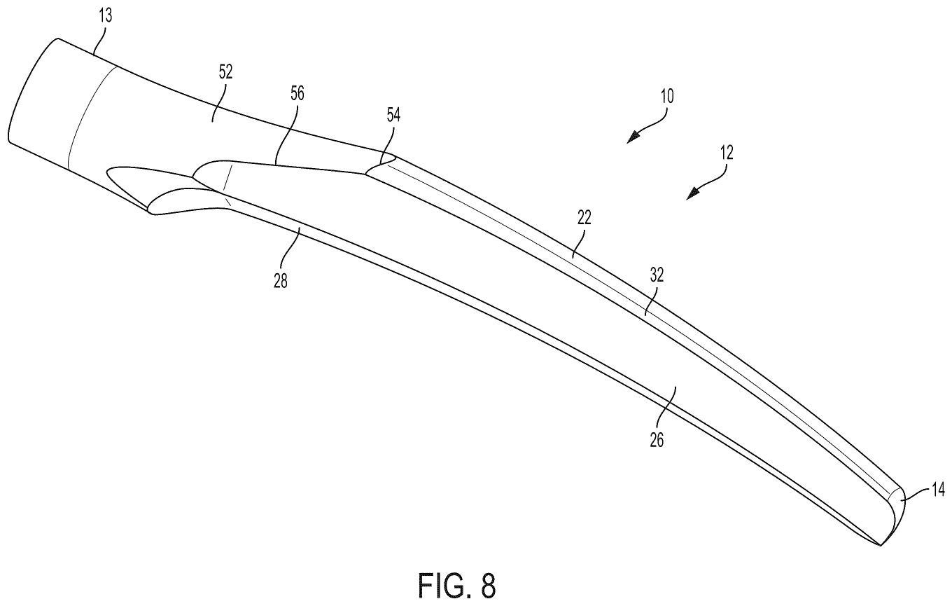

Referring generally to FIGS. 1-9, an ultrasonic blade 10 is depicted in various perspective views taken at approximately 30° intervals as the blade is rotated in the counterclockwise direction. The ultrasonic blade 10 comprises a body 12 that generally protrudes, or extends, from a base 13 in a distal direction terminating in a blunt, or at least substantially blunt, tip 14. The body 12 generally includes a first face 16 and a second face 18 opposite, or at least substantially opposite, the first face 16. The reader will appreciate that the blunt tip 14 can be fine but sufficiently atraumatic to allow for improved otomty creation and separation of tissue planes.

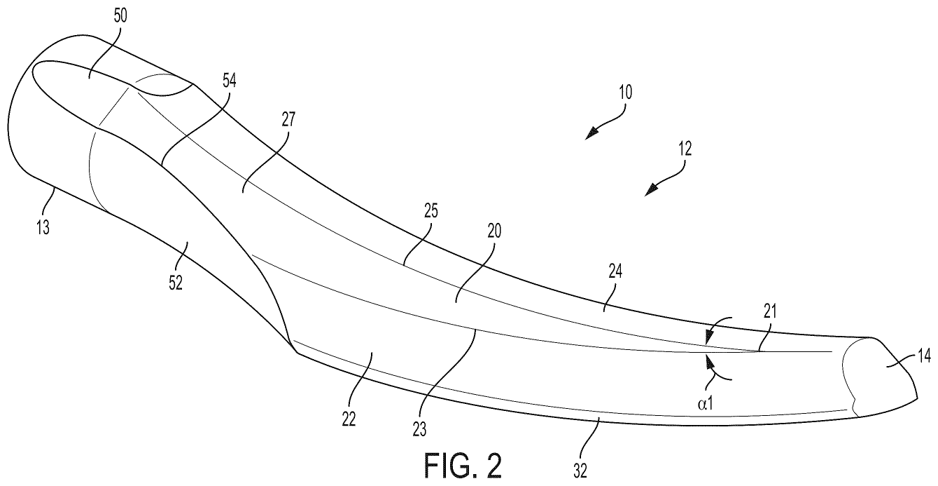

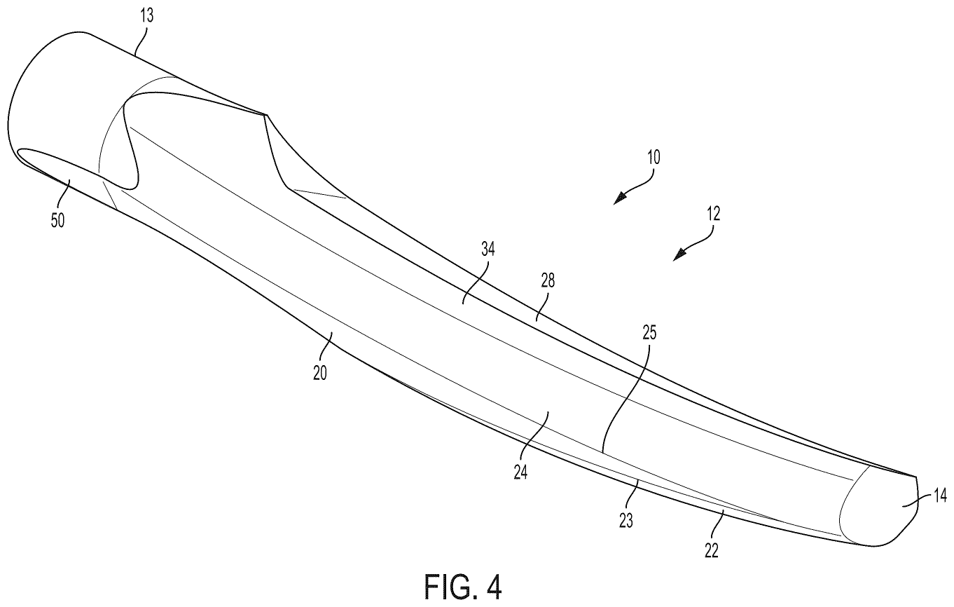

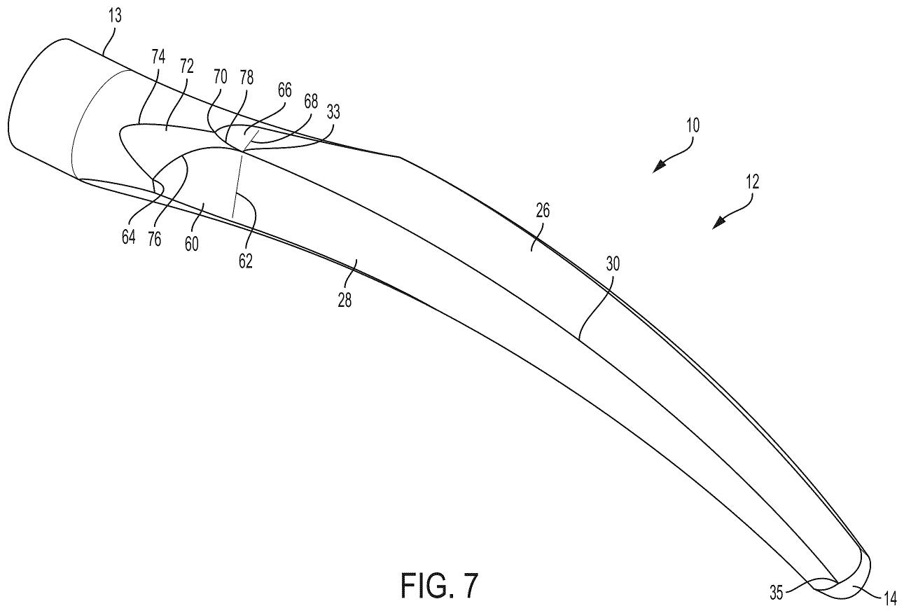

The first face 16 may include a tissue-treating surface 20, a first side wall 22, and a second side wall 24, as best illustrated in FIG. 2. The second face 18 may include a first tissue-cutting surface 26 and a second tissue-cutting surface 28 intersecting at a cutting edge 30, as best illustrated in FIG. 7. A first intermediate wall 32 may extend, at least partially, between the first side wall 22 and the first tissue-cutting surface 26, as best illustrated in FIG. 1. A second intermediate wall 34 may extend, at least partially, between the second side wall 24 and the second tissue-cutting surface 28, as best illustrated in FIG. 4.

The side walls 22, 24 extend, or substantially extend, on opposite sides of the tissue-treating surface 20, as best illustrated in FIG. 2. In one embodiment, as illustrated in FIG. 2, the tissue-treating surface 20 is beveled, or shaved, by the side walls 22, 24, which causes the tissue-treating surface 20 to be tapered, or narrowed, to a distal end 21 at a position proximal to the blunt tip 14. Said another way, the tissue-treating surface 20 is defined between two contour lines 23, 25 that extend proximally from the distal end 21. The first contour line 23 extends between the first side wall 22 and the tissue-treating surface 20. And, the second contour line 25 extends between the second side wall 24 and the tissue-treating surface 20. The contour lines 23, 25 follow separate and generally curved paths that intersect at the distal end 21.

The tissue-treating surface 20 can be flat, or at least substantially flat, such that the contour lines 23, 25 may extend, or substantially extend, in a single plane. The flatness of the tissue-treating surface 20 can facilitate using the tissue-treating surface 20 as a tissue sealing surface for sealing tissue captured against the tissue-treating surface 20. A significant advantage of the blade 10 is its ability to seal relatively large blood vessel such as, vessels comprising a diameter in the order of 7 mm, for example, with relatively short transection time such as, for example, 8 seconds. The one or both of the contour lines 23, 25 can be in the form of a sharp edge. Alternatively, one or both of the contour lines 23, 25 can be in the form of shaved to yield a smooth transition from the side walls 22, 24 to the tissue-treating surface 20.

In the remaining distance between the distal end 21 and the blunt tip 14, the side walls 22, 24 may directly intersect, or at least partially intersect, each other, for example. In other embodiments, the distal end 21 can be positioned at the blunt tip 14, or just proximal to the blunt tip 14. In at least one embodiment, the distal end 21 can be positioned closer to the blunt tip 15 than the base 13, as illustrated in FIG. 2. In at least one embodiment, the position of the distal end 21 can be equidistant between the blunt tip 14 and the base 13, for example.

An angle α1 can be defined between contour lines 23, 25, as illustrated in FIG. 2. The angle α1 can be any angle selected from a range of about 1° to about 15°, for example. In one example, the angle α1 can be any angle selected from a range of about 3° to about 10°. In one example, the angle α1 can be about 5°.

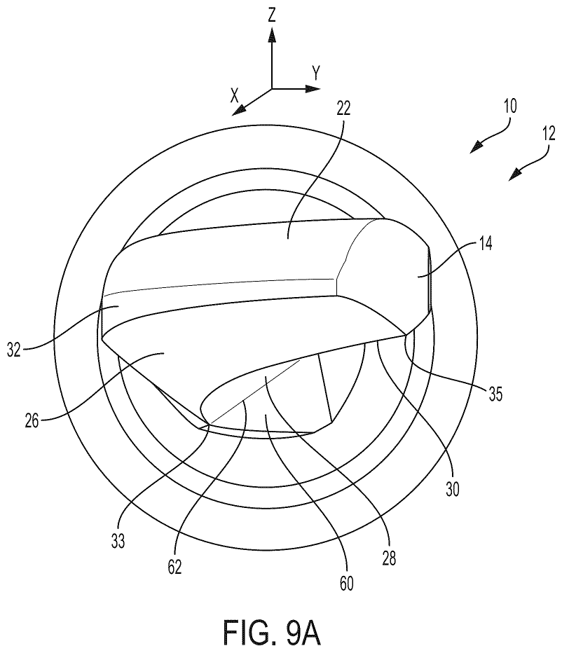

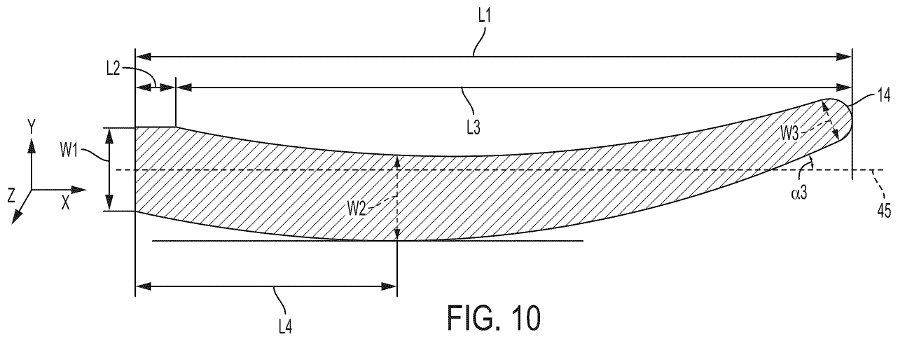

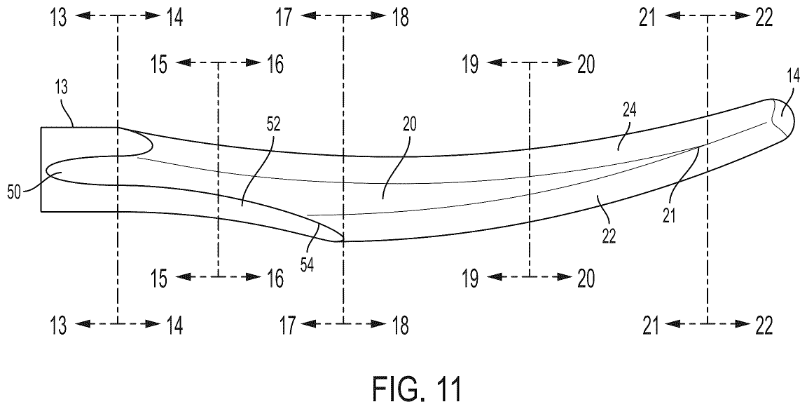

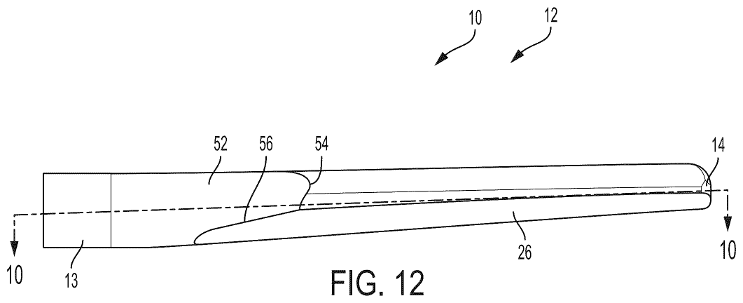

Referring to FIG. 10, a longitudinal cross-section of the blade 10 is depicted. The blade 10, as illustrated in FIG. 10, has a length L1, and includes a straight, or a substantially straight, portion 36 comprising a length L2, and a curved, or arcuate, portion 38 comprising a length L3. As illustrated in FIG. 10, the length L1 of the blade 10 is measured from the base 13 to the blunt tip 14. In certain instances, the length L1 can be any length selected from a range of about 0.4 inch to about 1.2 inch. In certain instances, the length L1 can be any length selected from a range of about 0.6 inch to about 0.8 inch. In certain instances, the length L1 can be any length selected from a range of about 0.70 inch to about 0.75 inch. In one instance, the length L1 can be about 0.728 inch, for example.

In certain instances, the length L2 can be any length selected from a range of about 0.02 inch to about 0.10 inch. In certain instances, the length L2 can be any length selected from a range of about 0.03 inch to about 0.07 inch. In one instance, the length L1 can be about 0.05 inch, for example.

In certain instances, the length L3 can be any length selected from a range of about 0.02 inch to about 0.10 inch. In certain instances, the length L2 can be any length selected from a range of about 0.03 inch to about 0.07 inch. In one instance, the length L3 can be about 0.767 inch, for example.

In certain instances, the length L3 can be any length selected from a range of about 0.50 inch to about 1.00 inch. In certain instances, the length L3 can be any length selected from a range of about 0.60 inch to about 0.80 inch. In one instance, the length L3 can be about 0.67 inch, for example. Other values for the lengths L1, L2, and/or L3 are contemplated by the present disclosure.

Referring again to FIG. 10, the blade 10 may comprise a width W1 at the base 13, a maximum width W2 at an inflection region 44, which is a distance L4 from the proximal end of the base 13, and a minimum width W3 at the blunt tip 14. As illustrated in FIG. 10, the width of the blade 10 can be tapered gradually along a length of the blade 10 from the maximum width W2 at the inflection region 44 to the minimum width W3 at the blunt tip 14, for example.

In certain instances, the width W1 can be any width selected from a range of about 0.030 inch to about 0.270 inch. In certain instances, the width W1 can be any width selected from a range of about 0.060 inch to about 0.180 inch. In one instance, the width W1 can be about 0.090 inch, for example.

In certain instances, the width W2 can be any width selected from a range of about 0.030 inch to about 0.270 inch. In certain instances, the width W2 can be any width selected from a range of about 0.060 inch to about 0.180 inch. In one instance, the width W2 can be about 0.090 inch, for example.

In certain instances, the width W3 can be any width selected from a range of about 0.120 inch to about 0.010 inch. In certain instances, the width W1 can be any width selected from a range of about 0.080 inch to about 0.030 inch. In one instance, the width W3 can be about 0.044 inch, for example. Other values for the widths W1, W2, and/or W3 are contemplated by the present disclosure.

In certain instances, the length L4 can be any length selected from a range of about 0.200 inch to about 0.300 inch. In certain instances, the length L4 can be any length selected from a range of about 0.250 inch to about 0.280 inch. In one instance, the length L3 can be about 0.270 inch, for example. Other values for the length L4 are contemplated by the present disclosure.

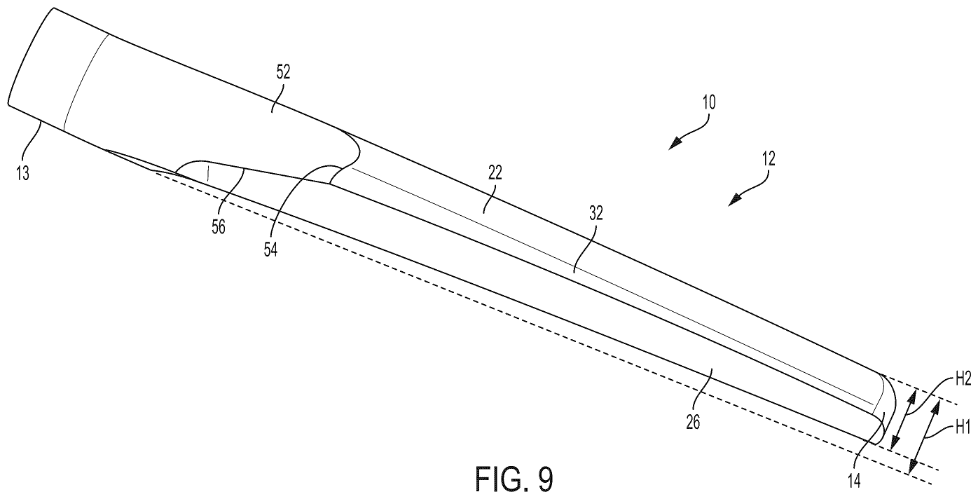

Referring primarily to FIGS. 9A, 10, Cartesian coordinate system is defined such that in FIG. 9A, the X-axis of the Cartesian coordinate system extends away from the page. And in FIG. 10, the Z-axis extends away from the page. In the embodiment illustrated in FIGS. 9A, 10, the X-and Z-axes of the Cartesian coordinate system define an XZ plane that transects the tissue-treating surface 20. In certain instances, as illustrated in 10, the XZ plane transects the tissue-treating surface 20 at an angle of about 90°, for example. In addition, the XZ plane may extend along a centerline 45 (FIG. 10) that extends through the base 13.

The curved portion 38 of the blade 10 is designed to fit and be passed through a tubular channel such as, for example, a trocar (not shown) during use of the blade 10 in a laparoscopic procedure, for example. Accordingly, to maximize the curvature of the blade 10 while staying within a size limit dictated by the diameter of the trocar, for example, the curved portion 38 of the blade 10 follows a unique trajectory with respect to the XZ plane.

Essentially, the curved portion 38 comprises a substantially arcuate profile that begins, or substantially begins, on a first side of the XZ plane, as illustrated in FIG. 10. The profile of the curved portion 38 then crosses the XZ plane to a second side of the XZ plane opposite the first side. Then, the profile of the curved portion 38 turns at an inflection region 44, and returns once again to the first side such that the blunt tip 14 is completely positioned on the first side of the XZ plane, as illustrated in FIG. 10. Accordingly, the profile of the curved portion 38 extends on both sides the XZ plane.

The cross-sectional area of the curved portion 38, as illustrated in FIG. 10, includes a proximal region 40, an inflection region 44, and a distal region 42. The proximal and distal regions 40, 42 can reside on the first side of the XZ plane, while the inflection region 44 resides mainly on the second side of the XZ plane.

In one example, more the 50% of the width W2 at the inflection region 44 resides on the second side of the XZ plane. In another example, more the 60% of the width W2 at the inflection region 44 resides on the second side of the XZ plane. In another example, more the 70% of the width W2 at the inflection region 44 resides on the second side of the XZ plane. In another example, more the 80% of the width W2 at the inflection region 44 resides on the second side of the XZ plane. In another example, more the 90% of the width W2 at the inflection region 44 resides on the second side of the XZ plane.

In certain instances, the curved portion 38 has a tilted or uneven curved profile. As illustrated in FIG. 10, the proximal and distal regions 40, 42 can reside at different perpendicular distances from the XZ plane. For example, the distal region 42 may be positioned further away from the XZ plane than the proximal region 40. Such a design is advantageous because it permits that blunt tip 14 of the blade 10 to extend or protrude outward, or to the side, beyond the base 13, which enhances the visibility of the blunt tip 14 from a position proximal to the base 13, as illustrated in FIG. 9A.

In the embodiment illustrated in FIG. 10, the distal region 42 intersects the centerline 45 at an angle α3 selected from a range of about 10° to about 80°, for example. In one embodiment, angle α3 can be any angle selected from a range of about 30° to about 60°, for example. In one embodiment, the angle α3 can be about 40°, for example. Other values for the angle α3 are contemplated by the present disclosure.

In one example, the perpendicular distance between the proximal region 40 and the XZ plane is about 0.045 inch, while the perpendicular distance between the distal region 42 and the XZ plane is about 0.075 inch. In this example, as illustrated in FIG. 10, the perpendicular distance between the inflection region 44 and the XZ plane is about 0.075 inch.

In certain instances, the perpendicular distance between the proximal region 40 and the XZ plane is any distance selected from a range of about 0.030 inch to about 0.080 inch. In certain instances, the perpendicular distance between the proximal region 40 and the XZ plane is any distance selected from a range of about 0.050 inch to about 0.060 inch. In certain instances, the perpendicular distance between the distal region 42 and the XZ plane is any distance selected from a range of about 0.050 inch to about 0.100 inch. In certain instances, the perpendicular distance between the distal region 42 and the XZ plane is any distance selected from a range of about 0.070 inch to about 0.080 inch. In certain instances, the perpendicular distance between the infliction region 44 and the XZ plane is any distance selected from a range of about 0.050 inch to about 0.100 inch. In certain instances, the perpendicular distance between the infliction region 44 and the XZ plane is any distance selected from a range of about 0.070 inch to about 0.080 inch. Other values for the distances described above are contemplated by the present disclosure.

In certain instances, the ratio of the perpendicular distance between the proximal region 40 and the XZ plane to the perpendicular distance between the distal region 42 and the XZ plane is any value selected from a range of about one quarter to about three quarters. In certain instances, the ratio of the perpendicular distance between the proximal region 40 and the XZ plane to the perpendicular distance between the distal region 42 and the XZ plane is any value selected from a range of about one third to about two thirds.

Like the width of the curved portion 38, the height of the curved portion 38 can be tapered as well. As illustrated in FIG. 9, the height of the curved portion 38 can be tapered gradually along a length of the blade 10 from a maximum height H1 at a the proximal end of the curved portion 38 to a minimum height H2 at the blunt tip 14, for example.

In certain instances, the height H1 can be any height selected from a range of about 0.070 inch to about 0.110 inch. In certain instances, the height H1 can be any height selected from a range of about 0.070 inch to about 0.100 inch. In one instance, the height H1 can be about 0.090 inch, for example.

In certain instances, the height H2 can be any height selected from a range of about 0.040 inch to about 0.070 inch. In certain instances, the height H2 can be any height selected from a range of about 0.050 inch to about 0.060 inch. In one instance, the height H2 can be about 0.054 inch, for example.

In certain instances, the ratio of the height H2 to the height H1 is selected from a range of values between about 0.2 to about 0.8. In certain instances, the ratio of the height H2 to the height H1 is selected from a range of values between about 0.3 to about 0.7. In one example, the ratio of the height H2 to the height H1 is about 0.6.

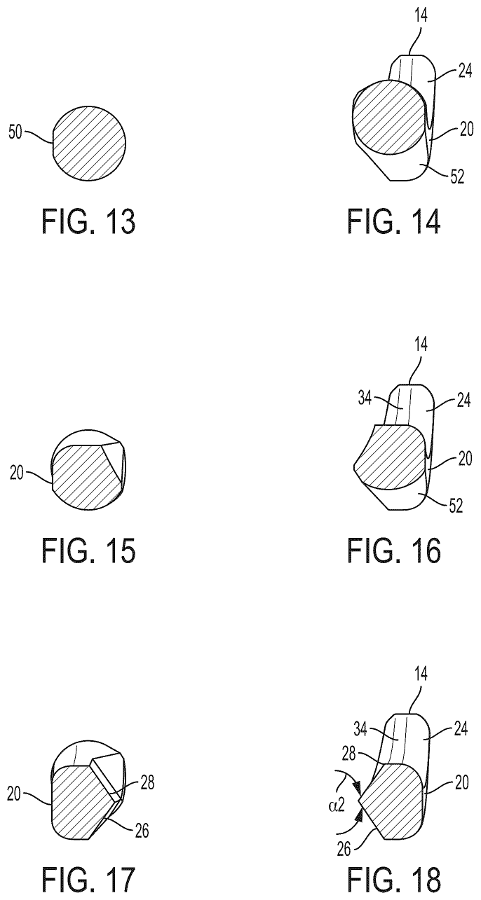

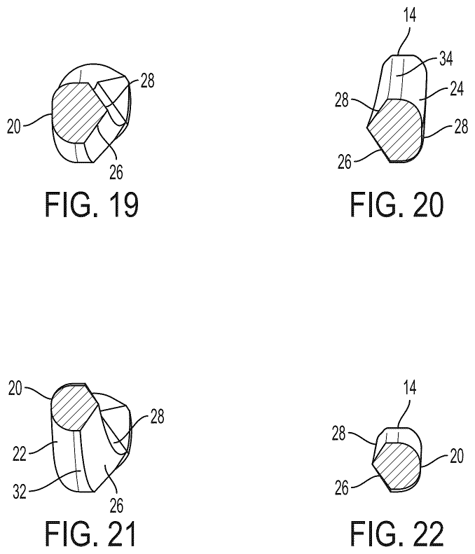

Referring primarily to FIGS. 1, 13, and 14, the base 13 may comprise a generally cylindrical shape with a flat top region 50 that extends proximally from the tissue-treating surface 20. As illustrated in FIG. 10, the centerline 45 may extend through a central axis of the cylindrical base 13. The curved portion 38 of the blade 10 crosses the centerline 45 causing the blunt tip 14 to extend, or protrude, laterally beyond the base 13 to enhance the visibility of the blunt tip 14 from a position proximal to the base 13, as illustrated in FIG. 14.

The blade 10 may include one or more acoustic balancing features. The acoustic balancing features are primarily sections of the blade 10 that are modified to acoustically balance the blade 10. In certain instances, the acoustically balancing features can be sections of the blade 10 that are removed or shaved to acoustically balance the blade 10.

Referring primarily to FIGS. 1, 15, and 16, a first acoustic balancing feature 52 is provided on a first side of the blade 10. The first acoustic balancing feature 52 encompasses a region that extends distally from a distal end of the base 13. In the embodiment illustrated in FIG. 1, the first acoustic balancing feature 52 has a first contour line 54 that intersects the tissue-treating surface 20, the first side wall 22, and first intermediate wall 32.

As illustrated in FIG. 1, the first contour line 54 of the acoustic balancing feature 52 interrupts the proximal extension of the of the first side wall 22 and the first intermediate wall 32 toward the base 13. A second contour line 56 of the first acoustic balancing feature 52 intersects the first tissue-cutting surface 26, as illustrated in FIG. 1. The contour lines 54 and 56 may intersect at a distal tip 57, as illustrated in FIG. 1.

As best illustrated in FIG. 2, the first contour line 54 of the first acoustic balancing feature 52 may interrupt the proximal extension of the first contour line 23 of the tissue-treating surface 20 thereby reducing or shaving the width of the tissue-treating surface 20 to create a narrowed proximal region 27. The interruptions caused by the first acoustic balancing feature 52 modify the shape of the blade 10 to improve its acoustic balance which is crucial to the function of the blade 10.

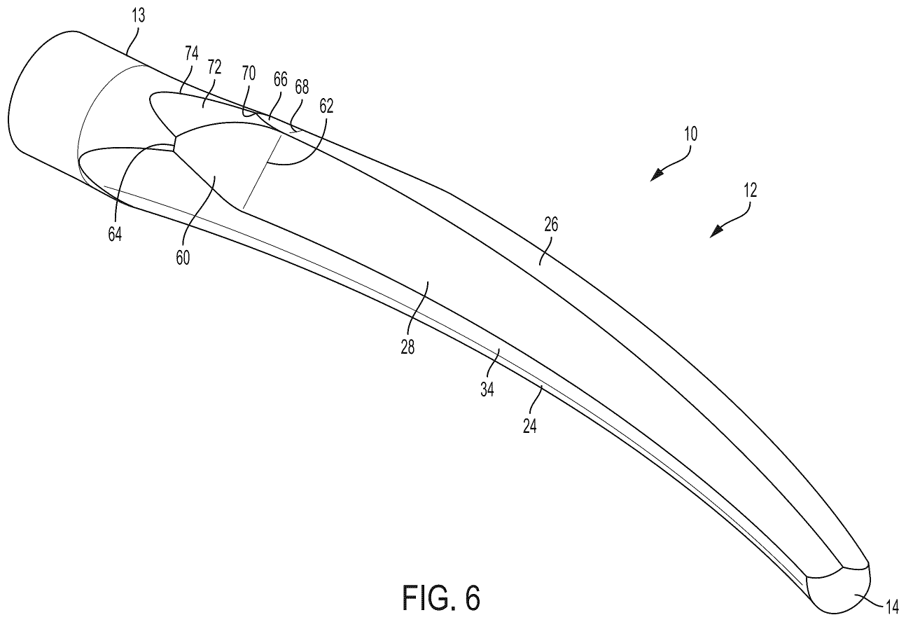

Referring primarily to FIG. 6, the blade 10 may include a second acoustic balancing feature 60. As illustrated in FIG. 6, the second acoustic balancing feature 60 may intersect the second tissue-cutting surface 28 at a contour line 62. In the embodiment illustrated in FIG. 6, the second acoustic balancing feature 60 comprises a generally curved shape that terminates at a peak 64.

Referring again to FIG. 6, a third acoustic balancing feature 66 may intersect the first tissue-cutting surface 26 at a contour line 68. Like the second acoustic balancing feature 60, the third acoustic balancing feature 66 may comprises a generally curved shape that terminates at a peak 70. In the embodiment illustrated in FIG. 6, the second acoustic balancing feature 60 comprises a greater radius of curvature that third acoustic balancing feature 66. In other embodiments, the third acoustic balancing feature 66 comprises a greater radius of curvature than the second acoustic balancing feature 60. In other embodiment the second acoustic balancing feature 60 and the third acoustic balancing feature 66 comprise the same, or at least substantially the same, radius of curvature.

Referring primarily to FIG. 7, a fourth acoustic balancing feature 72 extends further proximally from the second acoustic balancing feature 60 and the third acoustic balancing feature 66. The fourth acoustic balancing feature 72 includes a first contour line 74 which comprises a curved shape and generally extends from the peak 64 to the peak 70, as best illustrated in FIG. 7. Additionally, the fourth acoustic balancing feature 72 includes a second contour line 76 that intersects the second acoustic balancing feature 60, and a third contour line 78 that intersects the third acoustic balancing feature 66.

In the embodiment illustrated in FIG. 7, the contour lines 76, 78 intersect each other at a proximal end 33 of the cutting edge 30. Furthermore, the contour lines 62 and 68 may also intersect each other at the proximal end of the cutting edge 30, as illustrated in FIG. 7.

In one embodiment, the ultrasonic energy transmitted to tissue pressed against the cutting edge 30 can be employed to sever the tissue. In such embodiment, the blade 10 can be manipulated by an operator to position the cutting edge 30 against the tissue. Ultrasonic energy can then be transmitted to the tissue through the cutting edge 30 thereby causing the tissue to be severed or cut.

As best illustrated in FIG. 7, the first tissue cutting surface 26 extends distally from the contour line 68 which defines a distal perimeter of the third acoustic balancing feature 66. In a similar manner, the second tissue-cutting surface 28 extends distally from the contour line 62 which defines a distal perimeter of the second acoustic balancing feature 60. In the embodiment illustrated in FIG. 7, the cutting surfaces 26, 28 extend distally in generally curved paths along side each other defining a generally curved contour line therebetween the produces the cutting edge 30.

The reader will appreciate that the angle between the cutting surfaces 26, 28 can, at least in part, determine the sharpness of the cutting edge 30. In certain instances, the angle between the cutting surfaces 26, 28 can be the same, or substantially the same, along the length of the cutting edge 30. Alternatively, the angle between the cutting surfaces 26, 28 can be varied along the length, or at least a portion of the length, of the cutting edge 30. Accordingly, the sharpness of the cutting edge 30 can be varied along the length, or at least a portion of the length, of the cutting edge 30. In one embodiment, a proximal portion of the cutting edge 30 can be sharper than a distal portion of the cutting edge 30. Alternatively, a distal portion of the cutting edge 30 can be sharper than a proximal portion of the cutting edge 30.

The angle(s) between the cutting surfaces 26, 28 can be selected from a range of about 10° to about 175°. In one embodiment, the angle(s) between the cutting surfaces 26, 28 can be selected from a range of about 80° to about 170°. In one embodiment, the angle(s) between the cutting surfaces 26, 28 can be selected from a range of about 110° to about 160°. In one embodiment, the angle(s) between the cutting surfaces 26, 28 can be selected from a range of about 120° to about 150°. Other values for the angle(s) between the cutting surfaces 26, 28 can be contemplated by the present disclosure.

In one embodiment, referring primarily to FIG. 18, an angle α2 between the cutting surfaces 26, 28 at a distal end portion of the first acoustic balancing feature 52 may be selected from a range of about 85° to about 120°. In one instance, the angle α2 can be about 110°, for example.

Referring primarily to FIG. 9A, a front view of one embodiment of the blade 10 is illustrated. In the embodiment illustrated in FIG. 9A, the blade 10 is tapered such that the proximal end 33 of the cutting edge 30 is further away from a plane defined by the tissue-treating surface 20 than the distal end 35 of the cutting edge 30. Said another way, the proximal end 33 of the cutting edge 30 is vertically offset from the distal end 35 of the cutting edge 30.

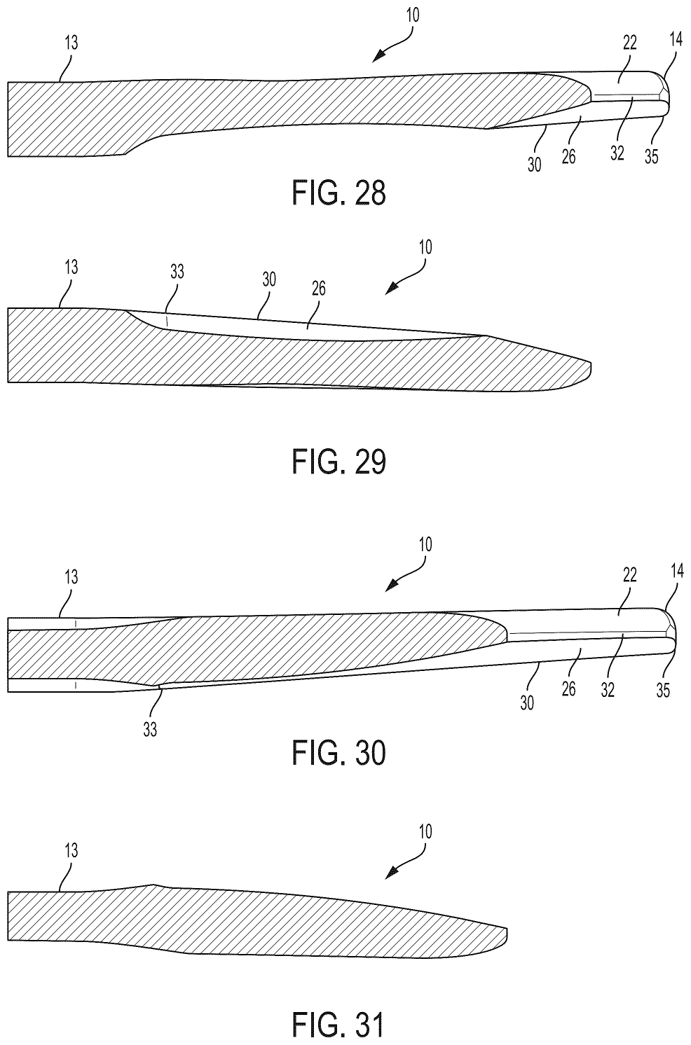

Furthermore, the proximal end 33 and the distal end 35 reside on opposite sides of the XZ plane. Said another way, the proximal end 33 of the cutting edge 30 can be horizontally offset from the distal end 35 of the cutting edge 30. FIGS. 28, 29 illustrate a longitudinal cross-section of the blade 10 taken along the XZ plane. FIG. 28 depicts the first side of the XZ plane which includes the distal end 35 of the cutting edge 30. FIG. 29 depicts the second side of the XZ plane which includes the proximal end 35 of the cutting edge 30.

In various instances, the ultrasonic blade 10 can be incorporated into an ultrasonic surgical instrument. For example, the ultrasonic blade 10 can be incorporated into an ultrasonic surgical instrument that includes a clamp member that may be controlled by an operator of the ultrasonic surgical instrument to capture tissue between the clamp member the ultrasonic blade 10 to treat the captured tissue. Examples of such ultrasonic surgical instruments and their mechanisms of operation are depicted in U.S. patent application Ser. No. 14/448,430, titled ACTUATION MECHANISMS AND LOAD ADJUSTMENT ASSEMBLIES FOR SURGICAL INSTRUMENTS, filed Jul. 31, 2014, which issued on May 14, 2019 as U.S. Pat. No. 10,285,724 and U.S. Patent Publication No. 2014/0005704 A1, titled ULTRASONIC SURGICAL INSTRUMENTS WITH DISTALLY POSITIONED JAW ASSEMBLIES, filed Jun. 29, 2012, which issued on May 31, 2016 as U.S. Pat. No. 9,351,754.

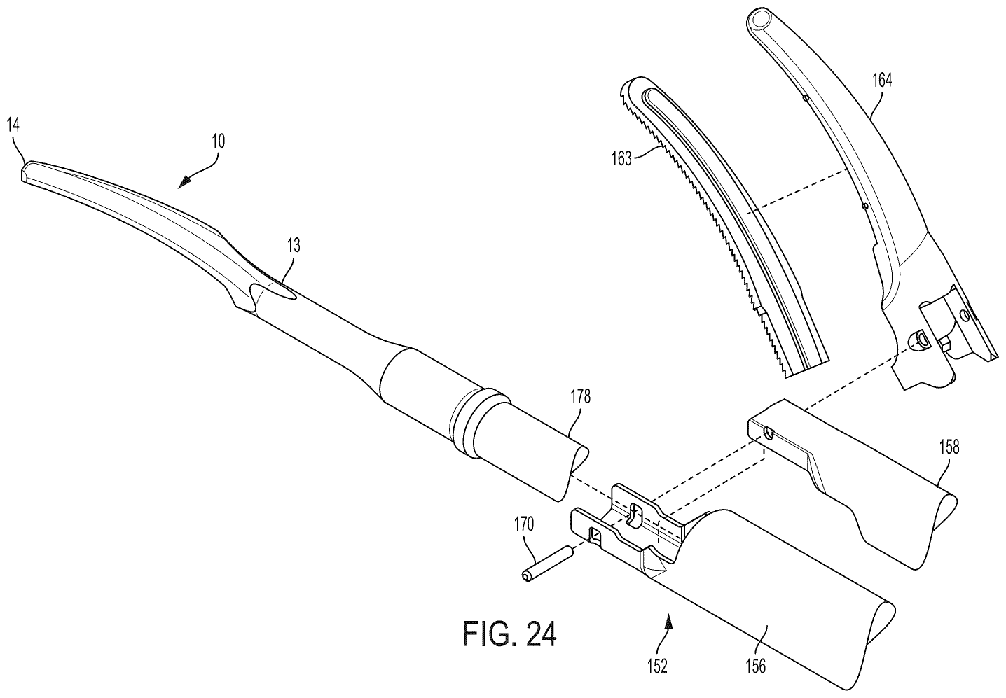

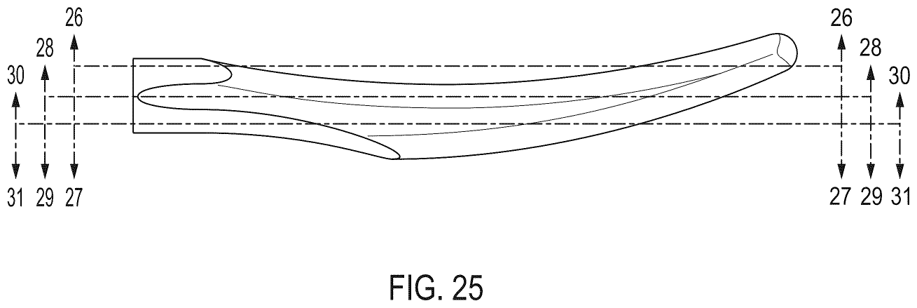

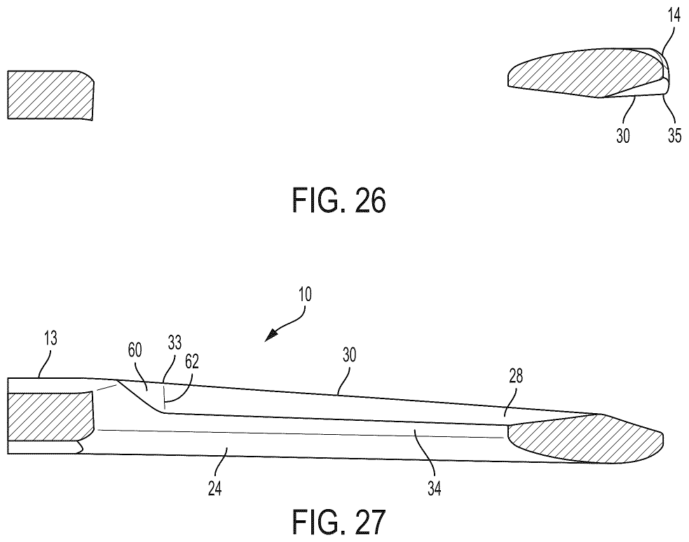

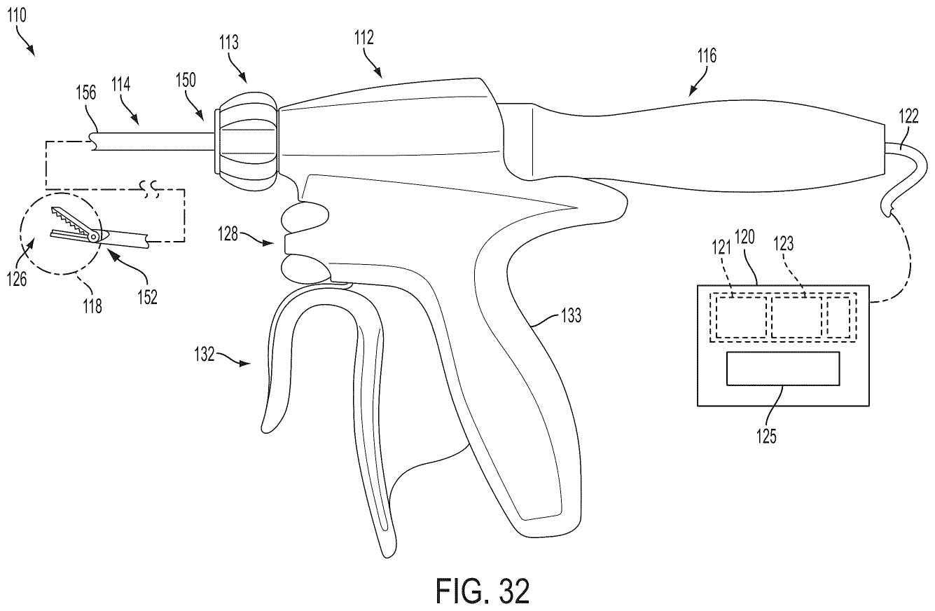

FIG. 32 illustrates a right side view of one embodiment of an ultrasonic surgical instrument 110 suitable for use with the ultrasonic blade 10. In the illustrated embodiment, the ultrasonic surgical instrument 110 may be employed in various surgical procedures including endoscopic or traditional open surgical procedures. In one example embodiment, the ultrasonic surgical instrument 110 comprises a handle assembly 112 extending proximally from an elongate shaft assembly 114, and an end effector assembly 126 extending distally from the elongate shaft assembly 114. An ultrasonic transmission waveguide 178 (FIG. 24) may extend, or at least partially extend, through the elongate shaft assembly 114. A distal end portion of the ultrasonic transmission waveguide 178 can be acoustically coupled (e.g., directly or indirectly mechanically coupled) to the blade 10. A proximal end portion of the ultrasonic transmission waveguide 178 can be received within the handle assembly 12 for acoustic coupling to an ultrasonic transducer 116.

The handle assembly 112 comprises a trigger 132, a handle 133, a distal rotation assembly 113, and a switch assembly 128. The elongated shaft assembly 114 comprises an end effector assembly 126 and actuating elements to actuate the end effector assembly 126. The handle assembly 112 is adapted to receive the ultrasonic transducer 116 at the proximal end. The ultrasonic transducer 116 can be mechanically engaged to the elongated shaft assembly 114 and portions of the end effector assembly 126. The ultrasonic transducer 116 can be electrically coupled to a generator 120 via a cable 122. In certain instances, the generator 120 can be integrated with the handle assembly 112, for example. A suitable generator is available as model number GEN11, from Ethicon Endo-Surgery, Inc., Cincinnati, Ohio.

The ultrasonic transducer 116 may convert the electrical signal from the ultrasonic signal generator 120 into mechanical energy that results in primarily a standing acoustic wave of longitudinal vibratory motion of the ultrasonic transducer 116 and the blade 10 portion of the end effector assembly 126 at ultrasonic frequencies.

In various instances, the energy generated in the blade 10 can be employed to cut and/or coagulate tissue. In one embodiment, vibrating at high frequencies (e.g., 55,500 times per second), the ultrasonic blade 10 may denature protein in the tissue to form a sticky coagulum.

Although the majority of the drawings depict a multiple end effector assembly 126 for use in connection with laparoscopic surgical procedures, the ultrasonic surgical instrument 110 may be employed in more traditional open surgical procedures and in other embodiments, may be configured for use in endoscopic procedures.

In various embodiments, the generator 20 comprises several functional elements, such as modules and/or blocks. Different functional elements or modules may be configured for driving different kinds of surgical devices. For example, an ultrasonic generator module 121 may drive an ultrasonic device, such as the ultrasonic surgical instrument 110. In some example embodiments, the generator 120 also comprises an electrosurgery/RF generator module 123 for driving an electrosurgical device. In the example embodiment illustrated in FIG. 1, the generator 120 includes a control system 125. When activated by the control system 125, the generator 120 may provide energy to drive the blade 10 of the surgical instrument 110.

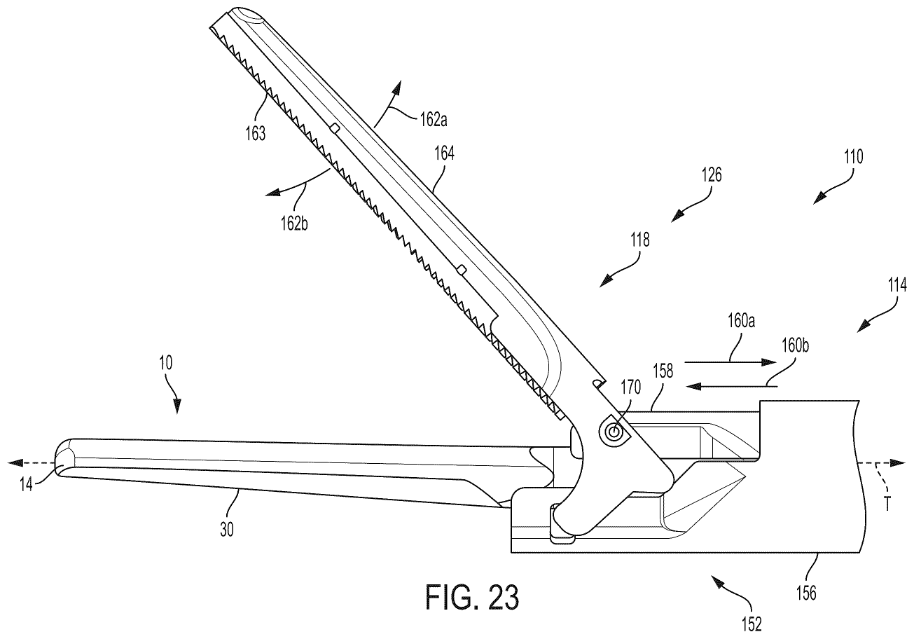

Referring to FIG. 32, the elongated shaft assembly 114 comprises a proximal end portion 150 adapted to mechanically engage the handle assembly 112 and the distal rotation assembly 113, and a distal end portion 152 adapted to mechanically engage the end effector assembly 126. The elongated shaft assembly 114 comprises an outer tubular sheath 156 and a reciprocating tubular actuating member 158 located within the outer tubular sheath 156, as illustrated in FIG. 23.

The proximal end of the tubular reciprocating tubular actuating member 158 is mechanically engaged to the trigger 132 of the handle assembly 112 to move in either direction 60A or 60B in response to the actuation and/or release of the trigger 132. The pivotably moveable trigger 132 may generate reciprocating motion along the longitudinal axis “T.” Such motion may be used, for example, to actuate an end effector 118 of the end effector assembly 126.

The distal end of the tubular reciprocating tubular actuating member 158 is mechanically engaged to the end effector 118 (FIG. 32). In the illustrated embodiment, the distal end of the tubular reciprocating tubular actuating member 158 is mechanically engaged to a clamp member 164, which is pivotable about a pivot point 170, to open and close the clamp member 164 in response to the actuation and/or release of the trigger 132. For example, in the illustrated embodiment, the clamp member 164 is movable in direction 162A from an open position to a closed position about a pivot point 170 when the trigger 132 is squeezed toward the handle 133. The clamp member 164 is movable in direction 162B from a closed position to an open position about the pivot point 170 when the trigger 132 is released.

In the closed position, tissue such as, for example, a blood vessel may be captured between the tissue-treating surface 20 of the blade 10 and a tissue grasping feature 163 of the clamp member 164 of the end effector 18. Pressure exerted on the captured tissue by the tissue-treating surface 20 may collapse the blood vessel and allow the coagulum resulting from application of the ultrasonic energy to form a hemostatic seal. A surgeon can control the cutting speed and coagulation by the force applied to the tissue by the end effector 18, the time over which the force is applied and the selected excursion level of the end effector 18.

The entire disclosures of:
U.S. patent application Ser. No. 14/448,430, titled ACTUATION MECHANISMS AND LOAD ADJUSTMENT ASSEMBLIES FOR SURGICAL INSTRUMENTS, filed Jul. 31, 2014, now U.S. Pat. No. 10,285,724; and
U.S. Patent Publication No. 2014/0005704 A1, titled ULTRASONIC SURGICAL INSTRUMENTS WITH DISTALLY POSITIONED JAW ASSEMBLIES, filed Jun. 29, 2012, now U.S. Pat. No. 9,351,754, are hereby incorporated by reference herein.

### Examples

Example 1—A surgical instrument, comprising an ultrasonic transducer, an ultrasonic transmission waveguide extending from the ultrasonic transducer, and an ultrasonic blade acoustically coupled to the ultrasonic transmission waveguide. The ultrasonic blade comprises a base and a curved body extending distally from the base. The curved body comprises, one, a tissue-treating surface extending on a first side of the curved body and, two, a curved edge extending on a second side of the curved body opposite the first side. The curved edge comprises a proximal end and a distal end, wherein the proximal end is offset from the distal end in a first direction, wherein the proximal end is offset from the distal end in a second direction, and wherein the first direction is perpendicular to the second direction.

Example 2—The surgical instrument of Example 1, further comprising a blunt tip, wherein the blunt tip extends laterally beyond the base.

Example 3—The surgical instrument of Example 2, wherein the tissue-treating surface is tapered toward a distal end proximal to the blunt tip.

Example 4—The surgical instrument of Examples 1, 2, or 3, further comprising a clamp member movable relative to the tissue-treating surface to capture tissue therebetween.

Example 5—The surgical instrument of Examples 1, 2, 3, or 4 further comprising a first acoustic balancing feature intersecting the tissue-treating surface.

Example 6—The surgical instrument of Example 5, further comprising second and third acoustic balancing features intersecting at the proximal end of the cutting edge.

Example 7—A surgical instrument, comprising an ultrasonic transducer, an ultrasonic transmission waveguide extending from the ultrasonic transducer, and an ultrasonic blade acoustically coupled to the ultrasonic transmission waveguide. The ultrasonic blade comprises a base and a curved body extending distally from the base. The curved body comprises a tissue-treating surface, a cutting edge, wherein the tissue-treating surface and the cutting edge are on opposite sides of the curved body, a blunt tip extending laterally beyond the base, a height gradually tapered toward the blunt tip, and a width gradually tapered toward the blunt tip.

Example 8—The surgical instrument of Example 7, further comprising a clamp member movable relative to the tissue-treating surface to capture tissue therebetween.

Example 9—The surgical instrument of Examples 7 or 8, wherein the tissue-treating surface is tapered toward a distal end proximal to the blunt tip.

Example 10—The surgical instrument of Examples 7, 8, or 9, further comprising a first acoustic balancing feature intersecting the tissue-treating surface.

Example 11—The surgical instrument of Example 10, further comprising second and third acoustic balancing features intersecting at a proximal end of the cutting edge.

Example 12—A surgical instrument, comprising an ultrasonic transducer, an ultrasonic transmission waveguide extending from the ultrasonic transducer, and an ultrasonic blade acoustically coupled to the ultrasonic transmission waveguide. The ultrasonic blade comprises a base and a curved portion extending distally from the base. The curved portion comprises a first face, comprising a tissue-treating surface, a first side wall intersecting the tissue-treating surface, and a second side wall intersecting the tissue-treating surface. The curved portion further comprises a second face, wherein the first face and the second face are on opposite sides of the curved portion, and wherein the second face comprises a first intermediate wall intersecting the first side wall, a second intermediate wall intersecting the second side wall, and a cutting edge extending distally along a length of the curved portion. The second face further comprises a first tissue cutting surface and a second tissue cutting surface, wherein the first tissue cutting surface intersects the second tissue cutting surface at the cutting edge.

Example 13—The surgical instrument of Example 12, further comprising a blunt tip, wherein the blunt tip extends laterally beyond the base.

Example 14—The surgical instrument of Example 13, wherein the tissue-treating surface is tapered toward a distal end proximal to the blunt tip.

Example 15—The surgical instrument of Examples 12, 13, or 14, further comprising a clamp member movable relative to the tissue-treating surface to capture tissue therebetween.

Example 16—The surgical instrument of Examples 12, 13, 14, or 15, further comprising a first acoustic balancing feature intersecting the tissue-treating surface.

Example 17—The surgical instrument of Examples 12, 13, 14, 15, or 16, further comprising a second acoustic balancing feature extending proximally from the second tissue cutting surface.

Example 18—The surgical instrument of Example 17, further comprising a third acoustic balancing feature extending proximally from the first tissue cutting surface.

Example 19—The surgical instrument of Example 18, further comprising a fourth acoustic balancing feature, wherein the second acoustic balancing feature defines a first peak, wherein the third acoustic balancing feature defines a second peak, and wherein the fourth acoustic balancing feature extends proximally from the first and second peaks.

Example 20—The surgical instrument of Examples 12, 13, 14, 15, 16, 17, 18, or 19, wherein the first and second tissue cutting surfaces define an angle therebetween, wherein the angle is selected from a range of about 85° to about 120°.

Although the various embodiments of the devices have been described herein in connection with certain disclosed embodiments, many modifications and variations to those embodiments may be implemented. Also, where materials are disclosed for certain components, other materials may be used. Furthermore, according to various embodiments, a single component may be replaced by multiple components, and multiple components may be replaced by a single component, to perform a given function or functions. The foregoing description and following claims are intended to cover all such modification and variations.

The devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

By way of example only, aspects described herein may be processed before surgery. First, a new or used instrument may be obtained and when necessary cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a medical facility. A device also may be sterilized using any other technique known in the art, including but not limited to beta or gamma radiation, ethylene oxide, plasma peroxide, or steam.

While this invention has been described as having exemplary designs, the present invention may be further modified within the spirit and scope of the disclosure. This application is therefore intended to cover any variations, uses, or adaptations of the invention using its general principles.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

1-20. (canceled)

21. A surgical instrument, comprising:
an ultrasonic transducer;
an ultrasonic transmission waveguide extending from the ultrasonic transducer; and
an ultrasonic blade acoustically coupled to the ultrasonic transmission waveguide, wherein the ultrasonic blade comprises:
a base;
a longitudinal axis extending parallel through the base; and
a curved body extending distally from the base, wherein the curved body comprises:
a distal end offset from the longitudinal axis in a first direction and a second direction different than the first direction;
a proximal end opposite the distal end;
a first side;
a second side opposite the first side; and
a curved edge extending on the second side, wherein the curved edge comprises:
a first portion curving away from the longitudinal axis at a point adjacent the proximal end;
a second portion curving toward the longitudinal axis at a point adjacent the distal end; and
a balancing feature comprising a contour line that defines an asymmetry in the curved body.

22. The surgical instrument of claim 21, wherein the curved body further comprises:
a blunt tip adjacent the distal end and extending laterally beyond the base.

23. The surgical instrument of claim 21, wherein the curved body further comprises:
a tissue-treating surface extending on the first side.

24. The surgical instrument of claim 23, wherein the tissue-treating surface is tapered toward the distal end.

25. The surgical instrument of claim 23, wherein the tissue-treating surface is curved convexly.

26. The surgical instrument of claim 23, further comprising:
a clamp member movable relative to the tissue-treating surface to capture tissue therebetween.

27. The surgical instrument of claim 23, wherein the contour line is curved concavely and intersects the tissue-treating surface.

28. A surgical instrument, comprising:
an ultrasonic transducer;
an ultrasonic transmission waveguide extending from the ultrasonic transducer; and
an ultrasonic blade acoustically coupled to the ultrasonic transmission waveguide, wherein the ultrasonic blade comprises:
a base;
a longitudinal axis extending parallel through the base; and
a curved body extending distally from the base, wherein the curved body comprises:
a distal end offset from the longitudinal axis in a first direction and a second direction different than the first direction;
a proximal end opposite the distal end;
a first side;
a second side opposite the first side;
a blunt tip adjacent the distal end and extending laterally beyond the base;
a height gradually tapered toward the blunt tip;
a width gradually tapered toward the blunt tip; and
a curved edge extending on the second side, wherein the curved edge comprises:
a first portion curving away from the longitudinal axis at a point adjacent the proximal end;
a second portion curving toward the longitudinal axis at a point adjacent the distal end; and
a balancing feature comprising a contour line that defines an asymmetry in the curved body.

29. The surgical instrument of claim 28, wherein the curved body further comprises:
a tissue-treating surface extending on the first side.

30. The surgical instrument of claim 29, wherein the tissue-treating surface is tapered toward the distal end.

31. The surgical instrument of claim 29, wherein the tissue-treating surface is curved convexly.

32. The surgical instrument of claim 29, further comprising:
a clamp member movable relative to the tissue-treating surface to capture tissue therebetween.

33. The surgical instrument of claim 29, wherein the contour line is curved concavely and intersects the tissue-treating surface.

34. A surgical instrument, comprising:
an ultrasonic transducer;
an ultrasonic transmission waveguide extending from the ultrasonic transducer; and
an ultrasonic blade acoustically coupled to the ultrasonic transmission waveguide, wherein the ultrasonic blade comprises:
a base;
a longitudinal axis extending parallel through the base; and
a curved portion extending distally from the base, wherein the curved portion comprises:
a distal end;
a proximal end opposite the distal end;
a first face, comprising:
a tissue-treating surface;
a first side wall intersecting the tissue-treating surface; and
a second side wall intersecting the tissue-treating surface; and
a second face opposite sides the first face, wherein the second face comprises:
a first intermediate wall intersecting the first side wall;
a second intermediate wall intersecting the second side wall;
a cutting edge extending distally along a length of the curved portion, wherein the cutting edge comprises:
a first portion curving away from the longitudinal axis at a point adjacent the proximal end;
a second portion curving toward the longitudinal axis at a point adjacent the distal end; and
a balancing feature comprising a contour line that defines an asymmetry in the curved portion; and;
a first tissue cutting surface; and
a second tissue cutting surface intersecting the first tissue cutting surface at the cutting edge.

35. The surgical instrument of claim 34, wherein the tissue-treating surface is tapered toward the distal end.

36. The surgical instrument of claim 34, wherein the tissue-treating surface is curved convexly.

37. The surgical instrument of claim 34, further comprising:
a clamp member movable relative to the tissue-treating surface to capture tissue therebetween.

38. The surgical instrument of claim 34, wherein the contour line is curved concavely and intersects the tissue-treating surface.

39. The surgical instrument of claim 34, further comprising:
a first acoustic balancing feature extending proximally from the first tissue cutting surface and defining a first peak;
a second acoustic balancing feature extending proximally from the second tissue cutting surface and defining a second peak; and
a third acoustic balancing feature extending proximally from the first and second peaks.

40. The surgical instrument of claim 34, wherein the first and second tissue cutting surfaces define an angle therebetween, wherein the angle is selected from a range of about 85° to about 120°.
